(19) United States
(12) Patent Application Publication
(10) Pub. No.: US 2015/0175974 A1
(43) Pub. Date: Jun. 25, 2015

(54) PRODUCING SOLIDS AND RELATED MOTHER LIQUORS

(71) Applicant: Syngenta Participations AG, Basel (CH)

(72) Inventors: James Owen Forrest, Greensboro, NC (US); Neil George, Bracknell (GB)

(21) Appl. No.: 14/406,571

(22) PCT Filed: Jun. 11, 2013
(86) PCT No.: PCT/US2013/045179

§ 371 Date: Dec. 9, 2014

Related U.S. Application Data
(60) Provisional application No. 61/658,149, filed on Jun. 11, 2012.

(51) Int. Cl. C12N 7/00

(52) U.S. Cl. CPC C12N 7/00; C12N 2795/00051

(57) ABSTRACT

Methods of producing solids, mother liquors, and resultant solids are shown and described. In one embodiment, a method of producing solids comprises creating a mother liquor comprising an AI and a solvent by dissolving the AI in the solvent. Phage particles having binding domains, or binding portions thereof, selected to bind to the AI are added to the mother liquor. The AI is precipitated as solid particles and separated from the mother liquor.

### FIELD OF TECHNOLOGY

The present disclosure relates generally to producing solid materials. More particularly, the present disclosure relates to using phage particles, or binding portions thereof to produce solid active ingredients in way that influences production. The disclosure is also directed to active ingredients produced according to method disclosed herein and to related mother liquors.

### BACKGROUND

Phage particles, or binding domains thereof, which show strong binding characteristics to active ingredient (AI) particles, such as the pesticide thiamethoxam, have been previously disclosed by applicants. In WO2011/066362, for example, applicants disclose, inter alia, the ability of the phage particles to improve the stability of formulated products.

Applicants have also now surprisingly demonstrated that phage particles, or binding domains thereof, can also be used to influence AI solids formation, e.g. crystal growth, to benefit AI manufacture processes. For example, existing methods of manufacturing crystalline active ingredients may require significant milling procedures after production of the active ingredient to produce particles of the desired size. Applicants desire, among other things, ways to reduce milling requirements.

### SUMMARY OF EXEMPLARY EMBODIMENTS

The present disclosure is directed to, inter alia, numerous improvements in the art of solids formation including methods of producing solids, e.g. crystals, solids produced by methods disclosed herein, and mother liquors for use in producing solids.

In one embodiment, a method of producing solids comprises creating a mother liquor comprising an active ingredient (AI) and a solvent by dissolving the AI in the solvent. Phage particles having binding domains, or binding portions thereof, selected to bind to the AI are added to the mother liquor. The AI is precipitated as solid particles and separated from the mother liquor.

Using methods as disclosed herein, at least one property of the precipitated AI solid particles can be altered. For example, the precipitated AI solid particles may have an average particle size smaller than AI precipitated without using phage particles having binding domains, or binding portions thereof. In another example, the precipitated AI solid particles may have a different shape than AI precipitated without using phage particles having binding domains, or binding portions thereof.

In another embodiment, the disclosure is directed to AI solid particles formed by any of the methods disclosed herein, wherein the AI solid particles also include a plurality of phage particles having binding domains, or binding portions thereof, selected to bind to the AI.

In another embodiment, the disclosure is directed to a mother liquor for precipitating solid particles. The mother liquor comprises a solvent; an AI concentration in the range of 5 wt % to 70 wt %; and a concentration of phage particles having binding domains, or binding portions thereof, selected to bind to the AI.

The above summary was intended to summarize certain embodiments of the present disclosure. Methods, related mother liquors and solids will be set forth in more detail, along with examples demonstrating efficacy, in the figures and detailed description below. It will be apparent, however, that the detailed description is not intended to limit the present invention, the scope of which should be properly determined by the appended claims.

### BRIEF DESCRIPTION OF THE FIGURES

FIG. 1 shows optical microscopy images of an active ingredient crystallized in the presence of phage particles having binding domains selected to bind to the active ingredient.

FIG. 2 shows electron microscopy images of an active ingredient crystallized in the presence of phage particles having binding domains selected to bind to the active ingredient.

FIG. 3 exemplifies different facets of an active ingredient crystal.

FIG. 4 shows the effect of phage particles having binding domains selected to bind to an active ingredient on the kinetics of desupersaturation.

### DETAILED DESCRIPTION OF PREFERRED EMBODIMENTS

As used herein:

An “active ingredient” or “AI” includes a biologically active substance, for example, a substance that prevents, destroys, repels or mitigates a pest.

A “crystal” or “crystalline material” includes a solid material whose constituent atoms, molecules, or ions are arranged in an ordered pattern extending in all three spatial dimensions. Crystal or crystalline material as used herein is also considered to be inclusive of polymorphic crystal structures.

A colloidal material (also termed simply a “colloid”) includes active ingredient solid particles (also referred to herein as “AI solid particles”) in a liquid phase, wherein the properties of the material are dominated by inter-particle forces acting between the surfaces of adjacent particles. Exemplary inter-particle forces include electrostatic forces, van der Waals attractive forces, London dispersion forces, hydrophobic interactions, etc. AI solid particles diameter or dimension may vary from embodiment to embodiment. Exemplary AI solid particles have diameters chosen from about 10 nanometer (nm) to about 100 micron (μm), in other examples, from about 100 nm to about 10 μm.

An “improvement in colloidal stability” is an improvement as measured by at least one of Colloidal Stability Assay I, Colloidal Stability Assay II, Colloidal Stability Assay III, and Colloidal Stability Assay IV.

Colloidal Stability Assay I (Physical Stability Assay)

The AI solid particles are dispersed in a liquid medium at a concentration convenient for packaging, transportation or sale. A sample of this liquid concentrate is placed in a glass container and stored either at a fixed temperature (which may be at, above or below ambient), or is subjected to temperature cycling from below ambient to either ambient or above. After a suitable interval the container is allowed to equilibrate to ambient temperature and the physical properties are compared with those before storage. The properties of interest include one or more of the following: viscosity as measured by a Brookfield rheometer or by a cup-and-bob or parallel plate type rheometer; the median particle size as measured by dynamic light scattering; the presence of any sediment may be determined by manual probe or visual examination; the presence of any serum may be determined by visual examination.

Colloidal Stability Assay II (Rate of Sedimentation Assay)

The AI solid particles are dispersed in a liquid medium at a concentration convenient for packaging, transportation or sale. A sample of this liquid concentrate is placed in a sample tube and subjected to centrifugation at a controlled temperature. The rate of serum or sediment formation is measured continuously either by visible light or X-ray transmittance or by visible light scattering.

Colloidal Stability Assay III (Dilution Assay)

A concentrated sample of colloidal material is diluted in a liquid medium to a concentration suitable for application to control an unwanted organism. This diluted sample is placed in a glass measuring cylinder and inverted repeatedly until the liquid dispersion is homogeneous. The cylinder is left undisturbed and examined periodically over 1 hour to monitor any visible flocculation and the rates of serum and sediment formation. After 24 hours the cylinder is inverted repeatedly at about 0.5 Hz and the number of inversions needed to re-homogenize any sediment is recorded.

This test may be performed at ambient temperature or below. The liquid medium may be water of defined hardness, or a liquid fertilizer solution suitable for agriculture, or an organic solvent suitable for application. This test may also be performed on concentrated samples stored under conditions described above in the Physical Stability Assay as a further method to assess changes in colloidal dispersion.

Colloidal Stability Assay IV (Flocculation Assay)

A concentrated sample of colloidal material is diluted in a liquid medium to a concentration suitable for application to control an unwanted organism. This diluted sample is observed under light microscopy to monitor any tendency of the colloidal particles to collect into flocculations. This behavior may be quantified by digital image analysis.

A “high binding affinity” means that after repeated wash cycles as described in the Affinity Example below, the surface concentration of bound phage, or binding portions thereof, remains at least about 2.0×10¹³ pfu/m².

A “mid binding affinity” means that after repeated wash cycles as described in the Affinity Example below, the surface concentration of bound phage, or binding portions thereof, is from about 2.0×10¹¹ pfu/m² to about 2.0×10¹³ pfu/m².

A “low binding affinity” means that after repeated wash cycles as described in Affinity Example below, the surface concentration of bound phage is from about 2.0×10⁹ pfu/m² to about 2.0×10¹¹ pfu/m².

### Affinity Example

Samples of AI solid particle suspensions with bacteriophage bound to the surface may be prepared as follows: Three 20 mL samples of a 1 wt % suspension of AI solid particles are prepared in PBS buffer with 0.1 wt % Tween® 20 (Croda, Plc, East Yorkshire, England) to aid dispersion. One 20 mg aliquot of bacteriophage suspension is added to each of the AI solid particle suspensions and these preparations are allowed to equilibrate overnight on a roller-bed. Unbound bacteriophage are washed from the samples by five successive washes (pellet by centrifugation, aspirate supernatant, add back 40 mL of PBS buffer, re-suspend by shaking). After the final supernatant aspiration, the volume was restored to the original 20 mL with PBS buffer, leaving a sample with essentially no unbound bacteriophage.

The bound bacteriophage are released from the AI solid particle surfaces as follows: A 1 mL aliquot of sample with no unbound bacteriophage as described above is pelleted by centrifugation, the supernatant is aspirated, the pellet is re-suspended in 0.66 mL of 100 mM Glycine (pH2.2) by vortex, then incubated for 10 minutes on a rotary mixer. These samples are centrifuged again and the supernatants, which now contain only the released bacteriophage, are collected by aspiration and transferred to sample tubes containing 0.33 mL 1M Tris buffer (pH 8.0) to neutralize.

In order to titre the bacteriophage samples, the bacteriophage titre protocol described by New England Biolabs may be followed. For example, 1 ul of each new bacteriophage sample is used to create a dilution series in LB: 1E3; 1E6; 1E9; 1E10; 1E11. 10 ul of each dilution is used to inoculate 200 ul of ER2738 cells (OD600=0.45) in 1.5 ml eppendorf tubes at room temperature for 5 mins. After this incubation, the contents of the inoculation tube are mixed with molten Top Agarose at 45 degC and immediately poured onto the surface of an LB-Xgal/IPTG plate. Once cooled, the plates are inverted and incubated at 37 degC overnight. Titre plates are inspected the following day.

The total volumes recovered is 3 mls. The yield of phage in terms of mass, can be estimated based on the assumption that 1 ug of M13 is approx. equivalent to 3.76E10 pfu. The approx. molecular mass of M13 is 16.3 MDa. The mass of 1 Da is 1.66053873E-24 g. Hence, one phage particle=2.656861968E-17 g. Thus, 1 ug of M13=3.76E10 particles (pfu).

The surface concentration of recovered phage particle “binding portions” may be determined by quantifying recovered binding portions based on a standard method for determining protein concentration such as the Bradford assay. Total protein concentration and the protein molecular weight can be used to determine the number of protein molecules per unit area of available surface. Phage particle “binding portions” are considered “plaque forming units” (pfus), regardless of their ability to form plaques, for determining concentration herein. For example, 1 binding portion peptide=1 pfu.

A “biologically effective amount” means an amount sufficient to either activate or inhibit a measurable process in a target organism. Such effects may be toxic or therapeutic depending on, for example, the embodiment.

“wt %” means wt/wt % unless indicated otherwise.

An “AI particle homolog” means a particle or component capable of eliciting at least the same level of biding affinity (i.e. low, mid or high) for an AI crystal as the AI crystal itself. Exemplary AI particle homologs include AI particle complexes, particles having similar moieties, co-crystals, etc.

An “icosahedral morphology” means a viral capsid that is nearly-spherical or contains a capsid shell of identical repeating subunits. Phage exhibiting exemplary icosahedral morphologies include the family Leviviridae, Microviridae, Corticoviridae, Cystoviridae, and Tectiviridae.

A “complex morphology” means any viral capsid that is neither purely helical nor purely icosahedral and possibly possesses extra structures such as protein tails or complex outer walls. Phage exhibiting exemplary complex morphologies include the family Myoviridae, Podoviridea, Siphoviridae, and Plasmaviridae.

A “filamentous morphology” means a viral capsid stacked around a central axis forming a helical structure, often with a central cavity or hollow tube. Phage exhibiting exemplary filamentous morphologies include the family Inoviridae and Lipothrixviridae.

A “major coat protein” means a coat protein present in the highest copy number in a phage coat or capsid. An exemplary major coat protein of phage M13 includes P8. A “minor coat protein” includes coat proteins other than the major coat protein. Exemplary minor coat proteins of phage M13 include P3, P6, P7 and P9.

Phage particle “binding domains” or “binding portions thereof” include peptides comprising a binding domain selected to bind to an AI solid particle, wherein the binding domain may be fused to at least one stability-helper peptide. Stability-helper peptides in conjunction with the binding domain may facilitate a reduction in particle size as described herein. Stability-helper peptides may also provide an improvement in colloidal stability as measured by at least one of Colloidal Stability Assay I, Colloidal Stability Assay II, Colloidal Stability Assay III, and Colloidal Stability Assay IV. Exemplary stability-helper peptides include at least one of phage M13's P8, P3, P6, P7 or P9 coat proteins, but the skilled practitioner will recognize that hydrophilic peptides in general will serve as stability-helper peptides according to, for example, the principle that polymeric dispersants comprise both hydrophobic domains that adsorb to AI solid particles and hydrophilic domains that remain solvated. In other embodiments, “binding domains” or “binding portions thereof” may include a peptide binding domain, such as an isolated peptide binding domain without a stability-helper peptide. Such binding domains may comprise the entire peptide or a portion thereof. Such peptides may be hydrophobic, hydrophilic or amphiphilic.

An “excipient” includes rheology modifiers, biocides, electrolytes, humectants, polymers, adjuvants, conventional surfactants, conventional dispersants, freezing point depressants, dyes, pigments, emetics, alerting agents, bird-repellants, anti-counterfeiting agents, fragrances, odor-masking agents, anti-drift agents, weathering inhibitors, foaming and defoaming agents.

A “phage-display library” includes a collection of phage having DNA encoding peptide or protein variants ligated into at least one coat protein, e.g., the pill or pVIII genes. The incorporation of many different DNA variants or fragments into the pill or pVIII genes permits the generation of a library from which members of interest can be selected and isolated. Commercially available phage-display libraries, for example, include Ph.D.-7, Ph.D.-12, and Ph.D.-C7C, available from New England Biolabs (Ipswich, Mass.). Phage-display libraries may be constructed as desired for use in accordance with the present invention.

Phage particles having binding domains may also vary from mixture to mixture. For example, phage particles may include members of at least one morphological group chosen from icosahedral, complex and filamentous phage. The binding domains of the phage particles may similarly vary, but are often biologically-expressed as translational fusions with phage coat proteins. The length of binding domains and their binding affinity may vary from embodiment to embodiment. Exemplary binding domains will have lengths chosen from about 3 to about 20 or more amino acids and binding affinities chosen from at least one of low, mid and high. Exemplary phage particles include M13 phage having 7, 8, 9, 10, 11, 12, 13, 14 or 15 amino acid-long binding domains fused to their P3 coat protein, with the binding domains having at least a low level of binding affinity.

Suitable binding domains may be obtained using a phage-display library available from New England Biolabs (Ipswich, Mass.). In addition to using binding domains obtained by commercial phage-display libraries, numerous protein structural domains are capable of forming contacts with target surfaces to achieve affinity-interactions and may be used. Such protein structural domains include, for example, the following domains and fragments thereof: FAb; Fv; scFv; stAb; dAb; V_{HH}; IgNAR; CDRs; DARPin ankyrin-repeat proteins; anti-calins; antibody-mimics. The ability to form translational fusions is within the skill of a person in the art.

Still, binding domains for phage particles or binding portions thereof may be generated in other ways. By way of example, the crystal structure of an active ingredient may be determined experimentally by conventional X-ray scattering techniques and the faces of the external crystal planes modeled using simulation software. Polypeptides with high binding affinity to each of the exposed crystal faces may then identified, for example, by calculating the most energetically favored secondary and tertiary conformation of a given polypeptide in water, by calculating the orientation of this polypeptide to each crystal face that maximizes the binding energy between the polypeptide and crystal, and by allowing the polypeptide secondary and tertiary structures to flex to further maximize the binding energy. This or other algorithms may be repeatedly applied to polypeptides with different primary structures until a peptide of the desired binding affinity is identified. The polypeptide may be produced by expression in a convenient organism, in cell-free extracts, or by chemical synthesis as known in the art. By way of example, see Stephen B. H. Kent, Chemical Synthesis of Peptides and Proteins, Ann. Rev. Biochem., 57:957-89 (1988) or R. Bruce Merrifield, Solid Phase Peptide Synthesis. |The Synthesis of a Tetrapeptide, J. Am. Chem. Soc., 85:2149-54 (1963). Synthesized peptides may be used with phage particles or binding portions thereof.

As noted above, one embodiment of the disclosure includes a method of producing active ingredient (AI) solid particles. In this embodiment, the method comprises creating a mother liquor comprising an active ingredient (AI) and a solvent by dissolving the AI in the solvent. Sovent selection may vary from embodiment to embodiment being determined, at least in part, by the physical chemistry of the active ingredient. For example, British Crop Protection Council, The Pesticide Manual, (11^{th} ed. 1998) describes the solubility of numerous AIs in different solvents.

The amount of AI in the mother liquor may vary from embodiment to embodiment. In an exemplary embodiment, AI will be added to create a mother liquor having an AI concentration in the range of 5 wt % to 70 wt %. In many embodiments, AI will be present in the range of 20 wt % to 50 wt %. Further, it should be clear that the AI may arrive in the mother liquor in a number of ways. For example, the AI may be added as a molten to the mother liquor; the AI may be added as previously precipitated material, e.g. in dry or slurry form; the AI may be extracted into the mother liquor; the AI may be synthesized in the mother liquor, e.g. through the addition of AI precursors to the mother liquor that are then synthesized to create the AI as part of a reaction step within the mother liquor; etc.

In many examples it will be necessary to heat the mother liquor to achieve AI saturation levels in the ranges disclosed herein. Heating will depend on the solvent, the AI, and the desired AI concentration in the mother liquor, however, heating to a temperature in the range of 0° C. to 100° C. should be sufficient for most embodiments disclosed herein. More typically, heating will be to temperatures in the range of 20° C. to 60° C.

Phage particles having binding domains, or binding portions thereof, selected to bind to the AI are added to the mother liquor. The generation of phage particles having binding domains, or binding portions thereof, selected to bind to the AI is disclosed herein, and readily achievable to one having ordinary skill in the art using a phage-display library. Phage particles, or binding domains thereof, selected may include any combination of those having a high binding affinity, a mid binding affinity, and a low binding affinity. In many examples, phage particles, or binding portions thereof, will be added to the AI to create a concentration in the range of 0.01% to 75% of the AI concentration. In other examples, phage particles, or binding portions thereof, will be added to the AI to create a concentration in the range of 10% to 30% of the AI concentration.

There is no sequential limitation on when phage particles having binding domains, or binding portions thereof, are added to the mother liquor. For example, adding phage particles to the mother liquor may include any combination of: adding phage particles to a vat in which the mother liquor will be formed prior to the addition of the solvent to the vat; adding phage particles to the solvent prior to the addition of AI; and adding phage particles to a mixture of solvent and AI.

After creation of the mother liquor, the AI solid particles are formed by precipitation from the mother liquor. In many examples, precipitation will be achieved by cooling the mother liquor. For example, if creation of the mother liquor included heating the mother liquor, e.g. heating to a temperature in the range of 50° C. to 100° C. to achieve the desired level of saturation, cooling could include the removing the mother liquor from heat or reducing the level of heat applied to the mother liquor. In many examples, cooling may include cooling to a temperature in the range of 20° C. to 60° C. Further, in many examples to facilitate precipitation, it may be desirable to control the rate of cooling, e.g. such that cooling occurs at a rate of 1° C. to 5° C. per hour. Further, precipitation may also be facilitated by the agitation of the mother liquor, e.g. to reduce the accumulation of precipitated AI particles in the bottom of the vat. A variety of mechanical agitators may be used for agitation, e.g. any number of paddles or blades commercially available.

In some examples, particularly those where AI solubility does not increase with temperature, precipitation may also include at least one of adding an antisolvent, e.g. heptane, to the mother liquor for driving the AI from the solvent, and evaporating the solvent from the mother liquor.

In some examples, precipitation may also include seeding the mother liquor with a plurality of seed AI particles to provide nucleation sites for the AI as it precipitates from solution. Seed AI particles may be, for example, small AI particles, e.g. having diameters chosen from about 10 nanometer (nm) to about 100 micron (μm), and may be added in a variety of ways, e.g. in powdered form, in slurry form, in mill base, as precipitated AI produced according the methods disclosed herein, as batch residue, etc. The amount of seed AI added to facilitate precipitation may vary. In many examples, seed AI will be added at a concentration in the range of 0.02% to 10%.

After precipitation the AI solid particles may be separated from the mother liquor for use in a variety of end-use formulations. Separation may be achieved, for example, by draining any remaining liquid from the mother liquor vat, by extracting the precipitated material from the bottom of the mother liquor vat using an extraction tube, extracting a slurry of precipitated material, etc.

Using methods as disclosed herein, at least one property of the precipitated AI solid particle can be altered. For example, the precipitated AI solid particles may have a smaller mean diameter than AI solid particles produced using traditional methods. For example, mean diameters of AI solid particles disclosed herein may be in the range of about 10⁻⁸ to about 10⁻⁴ m even without the requirement for milling to achieve the desired particle size. Diameter of particles may be estimated based on dynamic light scattering (DLS) theory. Suitable DLS detectors may be obtained from Malvern Instruments Ltd. having an office in Malvern, UK.

Methods disclosed herein may also be used to control the shape of AI solid particles formed during precipitation. For example, the precipitated AI solid particles may have a different shape, e.g have a different facet length or width, than AI precipitated without using phage particles having binding domains, or binding portions thereof.

In one example, the precipitated AI may include a first crystalline growth site and the phage particles having binding domains, or binding portions thereof, added to the mother liquor may be selected to bind to the first crystalline growth site. Not to be limited to a mechanism, but binding is believed to inhibit growth of the crystal at the first crystalline growth site, thereby creating a second shape (S2) that differs from a first shape (S1), wherein S1 is the shape created by a process that differs in that it does not include the addition of phage particles having binding domains, or binding portions thereof, to the mother liquor. S1 may differ from S2 by defining at least one different facet from S2. For example, if S1 includes facet F1 and S2 includes corresponding facet F2, F1 may differ from F2 by having a different length or width. Differences may be in the range of 1.5× to 5×, for example. In some examples, e.g. where needle-shaped crystal formation is undesirable (such as the needles are prone to clogging application equipment) it may be desirable to inhibit crystal grown in the linear direction of needle formation. In other examples, e.g. where milling may by employed or where crystal growth is highly irregular using traditional technologies, it may be desirable to promote linear growth so that shearing can be used to create more regularly sized particles, e.g. those sheared perpendicular to the length of the crystal. Screening using microscopy may be used for example to characterize crystal growth sites. It should be clear that “first”, “second”, “1” and “2” as used herein to describe shapes and geometries is simply for antecedent basis and is not intended to limit spatially, temporally, etc.

Further, it should be clear that mother liquors as disclosed herein are for producing AI technical ingredients and are readily distinguishable from formulated commercial product. For example, in most embodiments, mother liquors as used herein will not include at least one, at least two, at least three, at least four, or all of the following non-active ingredients: a surfactant, a diluent, a wetting agent, a safener, an antifoaming agent, and an emulsifier. Such non-active ingredients are readily recognizable to those in the formulating arts.

In another embodiment, the disclosure is directed to precipitated and separated AI solid particles formed by methods disclosed herein. The precipitated and separated AI solid particles can subsequently be combined with non-active ingredients such as at least one of a surfactant, a diluent, a wetting agent, an adhesive, a binding agent, a safener, an antifoaming agent, and an emulsifier to create any number of useful formulations. Formulations include, for example, suspensions (e.g. having the precipitated AI solid particles dispersed in a liquid); emulsifiable concentrates (e.g. a liquid system having a solution of precipitated AI solid particles in a solvent); suspoemulsions, (e.g. an aqueous formulation containing both a suspended active phase and emulsion droplets, wherein the precipitated AI solid particles may be in either or both of the suspended active phase and the emulsion droplet); soluble liquid (e.g. the precipitated AI solid particles are dissolved in a liquid); wettable powders (e.g. the precipitated AI solid particles are combined onto a solid carrier); water dispersible granules (e.g. the precipitated AI solid particles are adsorbed onto a solid carrier); microemulsifiable concentrates (e.g. the precipitated AI solid particles are dissolved in a solvent and upon dilution, form a microemulsion); capsule suspensions (e.g. a liquid formulation containing small droplets of the precipitated AI solid particles that have been dissolved in a solvent with the outside surface of the solvent drop being covered by a thin polymer shell); etc.

In some examples, the precipitated AI solid particles may be washed prior to using for formulated products to remove phage particles having binding domains, or binding portions thereof, selected to bind to the AI. Washing may be with 100 mM Glycine (pH2.2), for example, but others may prefer to wash with other solutions to release phage particles having binding domains, or binding portions thereof.

In other examples, the precipitated AI solid particles may be added to the formulated products while also containing a plurality of plurality of phage particles having binding domains, or binding portions thereof, selected to bind to the AI. The use of such material may create formulated products with improved colloidal stability, for example.

In another embodiment, the disclosure is directed to a mother liquor for precipitating AI solid particles. The mother liquor may vary as disclosed above.

The AI chosen may vary from industry to industry. In the agrochemical industry, for example, the AI may include at least one of an acaricide, an algicide, an avicide, a bactericide, a fungicide, a herbicide, an insecticide, a molluscicide, a nematicide, a rodenticide, and a virucide.

Any of the following agrochemical AIs capable of being precipitated from a mother liquor may be suitable for methods, mother liquors and precipitated AI as disclosed herein.

For example, at least one acaricide may be chosen from a antibiotic acaricide, such as nikkomycins and thuringiensin; a macrocyclic lactone acaricide, such as tetranactin; a avermectin acaricide, such as abamectin, doramectin, eprinomectin, ivermectin, and selamectin; a milbemycin acaricide, such as milbemectin, milbemycin, oxime, and moxidectin; a bridged diphenyl acaricide, such as azobenzene, benzoximate, benzyl benzoate, bromopropylate, chlorbenside, chlorfenethol, chlorfenson, chlorfensulphide, chlorobenzilate, chloropropylate, cyflumetofen, DDT, dicofol, diphenyl sulfone, dofenapyn, fenson, fentrifanil, fluorbenside, proclonol, tetradifon, and tetrasul; a carbamate acaricide, such as benomyl, carbanolate, carbaryl, carbofuran, methiocarb, metolcarb, promacyl, and propoxur; a oxime carbamate acaricide, such as aldicarb, butocarboxim, oxamyl, thiocarboxime, and thiofanox; a carbazate acaricide, such as bifenazate; a dinitrophenol acaricide, such as binapacryl, dinex, dinobuton, dinocap, dinocap-4, dinocap-6, dinocton, dinopenton, dinosulfon, dinoterbon, DNOC; a formamidine acaricide, such as amitraz, chlordimeform, chloromebuform, formetanate, and formparanate; a mite growth regulator, such as clofentezine, cyromazine, diflovidazin, dofenapyn, fluazuron, flubenzimine, flucycloxuron, flufenoxuron, and hexythiazox; an organochlorine acaricide, such as bromocyclen, camphechlor, DDT, dienochlor, endosulfan, and lindane; an organophosphorus acaricide; an organophosphate acaricide, such as chlorfenvinphos, crotoxyphos, dichlorvos, heptenophos, mevinphos, monocrotophos, naled, TEPP, and tetrachlorvinphos; an organothiophosphate acaricide, such as amidithion, amiton, azinphos-ethyl, azinphos-methyl, azothoate, benoxafos, bromophos, bromophos-ethyl, carbophenothion, chlorpyrifos, chlorthiophos, coumaphos, cyanthoate, demeton, demeton-O, demeton-S, demeton-methyl, demeton-O-methyl, demeton-S-methyl, demeton-S-methylsulphon, dialifos, diazinon, dimethoate, dioxathion, disulfoton, endothion, ethion, ethoate-methyl, formothion, malathion, mecarbam, methacrifos, omethoate, oxydeprofos, oxydisulfoton, parathion, phenkapton, phorate, phosalone, phosmet, phoxim, pirimiphos-methyl, prothidathion, prothoate, pyrimitate, quinalphos, quintiofos, sophamide, sulfotep, thiometon, triazophos, trifenofos, and vamidothion; a phosphonate acaricide, such as trichlorfon; a phosphoramidothioate acaricide such as isocarbophos, methamidophos, and propetamphos; a phosphorodiamide acaricide, such as dimefox, mipafox, and schradan; an organotin acaricide, such as azocyclotin, cyhexatin, and fenbutatin oxide; a phenylsulfamide acaricide, such as dichlofluanid; a phthalimide acaricide, such as dialifos and to phosmet; a pyrazole acaricide, such as cyenopyrafen, fenpyroximate, and tebufenpyrad; a phenylpyrazole acaricide, such as acetoprole, fipronil, and vaniliprole; a pyrethroid acaricide; a pyrethroid ester acaricide, such as acrinathrin, bifenthrin, cyhalothrin, cypermethrin, alpha-cypermethrin, fenpropathrin, fenvalerate, flucythrinate, flumethrin, fluvalinate, tau-fluvalinate, and permethrin; a pyrethroid ether acaricide, such as halfenprox; a pyrimidinamine acaricide such as pyrimidifen; a pyrrole acaricide, such as chlorfenapyr; a quinoxaline acaricide, such as chinomethionat and thioquinox; a sulfite ester acaricide, such as propargite; a tetronic acid acaricide, such as spirodiclofen; a tetrazine acaricide, such as clofentezine and diflovidazin; a thiazolidine acaricide, such as flubenzimine and hexythiazox; a thiocarbamate acaricide, such as fenothiocarb; a thiourea acaricide, such as chloromethiuron and diafenthiuron; and an unclassified acaricide, such as acequinocyl, amidoflumet, arsenous oxide, closantel, crotamiton, cymiazole, disulfuram, etoxazole, fenazaflor, fenazaquin, fluacrypyrim, fluenetil, mesulfen, MNAF, nifluridide, pyridaben, sulfuram, sulfluramid, sulfur, and triarathene.

At least one algicide may be may be chosen from a benzalkonium chloride, bethoxazin, copper sulfate, cybutryne, dichlone, dichlorophen, diuron, endothal, fentin, hydrated lime, isoproturon, methabenzthiazuron, nabam, oxyfluorfen, quinoclamine, quinonamid, simazine, and terbutryn.

At least one avicide may be chosen from 4-aminopyridine, chloralose, endrin, fenthion, and strychnine.

At least one bactericide may be chosen from bronopol, copper hydroxide, cresol, dichlorophen, dipyrithione, dodicin, fenaminosulf, formaldehyde, hydrargaphen, 8-hydroxyquinoline sulfate, kasugamycin, nitrapyrin, octhilinone, oxolinic acid, oxytetracycline, probenazole, streptomycin, tecloftalam, and thiomersal.

At least one chemosterilants may be chosen from apholate, bisazir, busulfan, diflubenzuron, dimatif, hemel, hempa, metepa, methiotepa, methyl apholate, morzid, penfluoron, tepa, thiohempa, thiotepa, tretamine, and uredepa.

At least one herbicide may may be chosen from an amide herbicide, such as allidochlor, amicarbazone, beflubutamid, benzadox, benzipram, bromobutide, cafenstrole, CDEA, cyprazole, dimethenamid, dimethenamid-P, diphenamid, epronaz, etnipromid, fentrazamide, flucarbazone, flupoxam, fomesafen, halosafen, isocarbamid, isoxaben, napropamide, naptalam, pethoxamid, propyzamide, quinonamid, saflufenacil, and tebutam; an anilide herbicide, such as chloranocryl, cisanilide, clomeprop, cypromid, diflufenican, etobenzanid, fenasulam, flufenacet, flufenican, ipfencarbazone, mefenacet, mefluidide, metamifop, monalide, naproanilide, pentanochlor, picolinafen, propanil, and sulfentrazone; an arylalanine herbicide, such as benzoylprop, flamprop, and flamprop-M; a chloroacetanilide herbicide, such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, and xylachlor; a sulfonanilide herbicide, such as benzofluor, cloransulam, diclosulam, florasulam, flumetsulam, metosulam, perfluidone, pyrimisulfan, and profluazol; a sulfonamide herbicide, such as asulam, carbasulam, fenasulam, oryzalin, penoxsulam, pyroxsulam; a thioamide herbicide, such as bencarbazone and chlorthiamid; an antibiotic herbicide, such as bilanafos; an aromatic acid herbicide; a benzoic acid herbicide, such as chloramben, dicamba, 2,3,6-TBA and tricamba; a pyrimidinyloxybenzoic acid herbicide, such as bispyribac and pyriminobac; a pyrimidinylthiobenzoic acid herbicide, such as pyrithiobac; a phthalic acid herbicide, such as chlorthal; a picolinic acid herbicide, such as aminopyralid, clopyralid, and picloram; a quinolinecarboxylic acid herbicide, such as quinclorac, and quinmerac; an arsenical herbicide, such as cacodylic acid, CMA, DSMA, hexaflurate, MAA, MAMA, MSMA, potassium arsenite, and sodium arsenite; a benzoylcyclohexanedione herbicide, such as mesotrione, sulcotrione, tefuryltrione, and tembotrione; a benzofuranyl alkylsulfonate herbicide, such as benfuresate and ethofumesate; a benzothiazole herbicide, such as benazolin, benzthiazuron, fenthiaprop, mefenacet, and methabenzthiazuron; a carbamate herbicide, such as asulam, carboxazole, chlorprocarb, dichlormate, fenasulam, karbutilate, and terbucarb; a carbanilate herbicide, such as barban, BCPC, carbasulam, carbetamide, CEPC, chlorbufam, chlorpropham, CPPC, desmedipham, phenisopham, phenmedipham, phenmedipham-ethyl, propham and swep; a cyclohexene oxime herbicide, such as alloxydim, butroxydim, clethodim, cloproxydim, cycloxydim, profoxydim, sethoxydim, tepraloxydim, and tralkoxydim; a cyclopropylisoxazole herbicide, such as isoxachlortole and isoxaflutole; a dicarboximide herbicide, such as cinidon-ethyl, flumezin, flumiclorac, flumioxazin, and flumipropyn; a dinitroaniline herbicide, such as benfluralin, butralin, dinitramine, ethalfluralin, fluchloralin, isopropalin, methalpropalin, nitralin, oryzalin, pendimethalin, prodiamine, profluralin, and trifluralin; a dinitrophenol herbicide, such as dinofenate, dinoprop, dinosam, dinoseb, dinoterb, DNOC, etinofen, and medinoterb; a diphenyl ether herbicide, such as ethoxyfen; a nitrophenyl ether herbicide, such as acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen, and oxyfluorfen; a dithiocarbamate herbicide, such as dazomet and metam; a halogenated aliphatic herbicide, such as alorac, chloropon, dalapon, flupropanate, hexachloroacetone, iodomethane, methyl bromide, monochloroacetic acid, SMA, and TCA; a imidazolinone herbicide, such as imazamethabenz, imazamox, imazapic, imazapyr, imazaquin, and imazethapyr; an inorganic herbicide, such as ammonium sulfamate, borax, calcium, hlorate, copper sulfate, ferrous sulfate, potassium azide, potassium cyanate, sodium azide, sodium chlorate, and sulfuric acid; a nitrile herbicide, such as bromobonil, bromoxynil, chloroxynil, dichlobenil, iodobonil, ioxynil, and pyraclonil; an organophosphorus herbicide, such as amiprofos-methyl, anilofos, bensulide, bilanafos, butamifos, 2,4-DEP, DMPA, EBEP, fosamine, glufosinate, glufosinate-P, glyphosate, and piperophos; an oxadiazolone herbicide, such as dimefuron, methazole, oxadiargyl, and oxadiazon; an oxazole herbicide, such as carboxazole, fenoxasulfone, isouron, isoxaben, isoxachlortole, isoxaflutole, monisouron, pyroxasulfone, and topramezone; a phenoxy herbicide, such as bromofenoxim, clomeprop, 2,4-DEB, 2,4-DEP, difenopenten, disul, erbon, etnipromid, fenteracol, and trifopsime; a phenoxyacetic herbicide, such as 4-CPA, 2,4-D, 3,4-DA, MCPA, MCPA-thioethyl, and 2,4,5-T; a phenoxybutyric herbicide, such as 4-CPB, 2,4-DB, 3,4-DB, MCPB, and 2,4,5-TB, a phenoxypropionic herbicide, such as cloprop, 4-CPP, dichlorprop, dichlorprop-P, 3,4-DP, fenoprop, mecoprop, and mecoprop-P; an aryloxyphenoxypropionic herbicide, such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, and trifop; a phenylenediamine herbicide, such as dinitramine and prodiamine; a pyrazole herbicide, such as azimsulfuron, difenzoquat, halosulfuron, metazachlor, metazosulfuron, pyrazosulfuron, and pyroxasulfone; a benzoylpyrazole herbicide, such as benzofenap, pyrasulfotole, pyrazolynate, pyrazoxyfen, and topramezone; a to phenylpyrazole herbicide, such as fluazolate, nipyraclofen, pinoxaden, and pyraflufen; a pyridazine herbicide, such as credazine, pyridafol, and pyridate; a pyridazinone herbicide, such as brompyrazon, chloridazon, dimidazon, flufenpyr, metflurazon, norflurazon, oxapyrazon, and pydanon; a pyridine herbicide, such as aminopyralid, cliodinate, clopyralid, diflufenican, dithiopyr, flufenican, fluoroxypyr, haloxydine, picloram, picolinafen, pyriclor, pyroxsulam, thiazopyr, and triclopyr; a pyrimidinediamine herbicide, such as iprymidam and tioclorim, a quaternary ammonium herbicide, such as cyperquat, diethamquat, difenzoquat, diquat, morfamquat, and paraquat; a thiocarbamate herbicide, such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, and vernolate; a thiocarbonate herbicide, such as dimexano, EXD, and proxan; a thiourea herbicide, such as methiuron; a triazine herbicide, such as dipropetryn, indaziflam, triaziflam, and trihydroxytriazine; a chlorotriazine herbicide, such as atrazine, chlorazine, cyanazine, cyprazine, eglinazine, ipazine, mesoprazine, procyazine, proglinazine, propazine, sebuthylazine, simazine, terbuthylazine, and trietazine; a methoxytriazine herbicide, such as atraton, methometon, prometon, secbumeton, simeton, and terbumeton; a methylthiotriazine herbicide, such as ametryn, aziprotryne, cyanatryn, desmetryn, dimethametryn, methoprotryne, prometryn, simetryn, and terbutryn; a triazinone herbicide, such as ametridione, amibuzin, hexazinone, isomethiozin, metamitron and metribuzin; a triazole herbicide, such as amitrole, cafenstrole, epronaz, and flupoxam; a triazolone herbicide, such as amicarbazone, bencarbazone, carfentrazone, flucarbazone, ipfencarbazone, propoxycarbazone, sulfentrazone, and thiencarbazone; a triazolopyrimidine herbicide, such as cloransulam, diclosulam, florasulam, flumetsulam, metosulam, penoxsulam, and pyroxsulam; an uracil herbicide, such as benzfendizone, bromacil, butafenacil, flupropacil, isocil, lenacil, saflufenacil, and terbacil; an urea herbicide, such as benzthiazuron, cumyluron, cycluron, dichloralurea, diflufenzopyr, isonoruron, isouron, methabenzthiazuron, monisouron, noruron, a phenylurea herbicide, such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron, and thidiazuron; a sulfonylurea herbicide; a pyrimidinylsulfonylurea herbicide, such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, metazosulfuron, nicosulfuron, orthosulfamuron, oxasulfuron, primisulfuron, propyrisulfuron, pyrazosulfuron, rimsulfuron, sulfometuron, sulfosulfuron, and trifloxysulfuron; a triazinylsulfonylurea herbicide, such as chlorsulfuron, cinosulfuron, ethametsulfuron, iodosulfuron, metsulfuron, prosulfuron, thifensulfuron, triasulfuron, tribenuron, triflusulfuron, and tritosulfuron; a thiadiazolylurea herbicide, such as buthiuron, ethidimuron, tebuthiuron, thiazafluoron, and thidiazuron; and an unclassified herbicide, such as acrolein, allyl alcohol, aminocyclopyrachlor, azafenidin, bentazone, benzobicyclon, bicyclopyrone, buthidazole, calcium cyanamide, cambendichlor, chlorfenac, chlorfenprop, chlorflurazole, chlorflurenol, cinmethylin, clomazone, CPMF, cresol, cyanamide, ortho-dichlorobenzene, dimepiperate, endothal, fluoromidine, fluridone, fluorochloridone, flurtamone, fluthiacet, indanofan, methyl isothiocyanate, OCH, oxaziclomefone, pentachlorophenol, pentoxazone, phenylmercury acetate, prosulfalin, pyribenzoxim, pyriftalid, quinoclamine, rhodethanil, sulglycapin, thidiazimin, tridiphane, trimeturon, tripropindan, and tritac.

At least one fungicide may be chosen from an aliphatic nitrogen fungicide, such as butylamine, cymoxanil, dodicin, dodine, guazatine, iminoctadine; an amide fungicide, such as carpropamid, chloraniformethan, cyflufenamid, diclocymet, ethaboxam, fenoxanil, flumetover, furametpyr, isopyrazam, mandipropamid, penthiopyrad, prochloraz, quinazamid, silthiofam, and triforine; an acylamino acid fungicide, such as benalaxyl, benalaxyl-M, furalaxyl, metalaxyl, metalaxyl-M, pefurazoate, and valifenalate; an anilide fungicide, such as benalaxyl, benalaxyl-M, bixafen, boscalid, carboxin, fenhexamid, isotianil, metalaxyl, metalaxyl-M, metsulfovax, ofurace, oxadixyl, oxycarboxin, penflufen, pyracarbolid, sedaxane, thifluzamide, and tiadinil; a benzanilide fungicide, such as benodanil, flutolanil, mebenil, mepronil, salicylanilide, and tecloftalam; a furanilide fungicide, such as fenfuram, furalaxyl, furcarbanil, and methfuroxam; a sulfonanilide fungicide, such as flusulfamide; a benzamide fungicide, such as benzohydroxamic acid, fluopicolide, fluopyram, tioxymid, trichlamide, zarilamid, and zoxamide; a furamide fungicide, such as cyclafuramid and furmecyclox; a phenylsulfamide fungicide, such as dichlofluanid and tolylfluanid; a sulfonamide fungicide, such as amisulbrom and cyazofamid; a valinamide fungicide, such as benthiavalicarb and iprovalicarb; an antibiotic fungicide, such as aureofungin, blasticidin-S, cycloheximide, griseofulvin, kasugamycin, natamycin, polyoxins, polyoxorim, streptomycin, and validamycin; a strobilurin fungicide, such as azoxystrobin, dimoxystrobin, fluoxastrobin, kresoxim-methyl, metominostrobin, orysastrobin, picoxystrobin, pyraclostrobin, pyrametostrobin, pyraoxystrobin, and trifloxystrobin; an aromatic fungicide, such as biphenyl, chlorodinitronaphthalene, chloroneb, chlorothalonil, cresol, dicloran, hexachlorobenzene, pentachlorophenol, quintozene, sodium pentachlorophenoxide, and tecnazene; a benzimidazole fungicide, such as benomyl, carbendazim, chlorfenazole, cypendazole, debacarb, fuberidazole, mecarbinzid, rabenzazole, and thiabendazole; a benzimidazole precursor fungicide, such as furophanate, thiophanate, and thiophanate-methyl; a benzothiazole fungicide, such as bentaluron, benthiavalicarb, chlobenthiazone, probenazole, and TCMTB; a bridged diphenyl fungicide, such as bithionol, dichlorophen, and diphenylamine; a carbamate fungicide, such as benthiavalicarb, furophanate, iprovalicarb, propamocarb, pyribencarb, thiophanate, and thiophanate-methyl; a benzimidazolylcarbamate fungicide, such as benomyl, carbendazim, cypendazole, debacarb, and mecarbinzid; a carbanilate fungicide, such as diethofencarb, pyraclostrobin, and pyrametostrobin; a conazole fungicide; a conazole fungicide (imidazoles), such as climbazole, clotrimazole, imazalil, oxpoconazole, prochloraz, triflumizole; a conazole fungicide (triazoles), such as azaconazole, bromuconazole, cyproconazole, diclobutrazol, difenoconazole, diniconazole diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P; a copper fungicide, such as Bordeaux mixture, Burgundy mixture, Cheshunt mixture, copper acetate, copper carbonate, basic, copper hydroxide, copper naphthenate, copper oleate, copper oxychloride, copper silicate, copper sulfate, copper sulfate, basic, copper zinc chromate, cufraneb, cuprobam, cuprous oxide, mancopper, and oxine-copper; a dicarboximide fungicide, such as famoxadone and fluoroimide; a dichlorophenyl dicarboximide fungicide, such as chlozolinate, dichlozoline, iprodione, isovaledione, myclozolin, procymidone, and vinclozolin; a phthalimide fungicide, such as captafol, captan, ditalimfos, folpet, and thiochlorfenphim; a dinitrophenol fungicide, such as binapacryl, dinobuton, dinocap, dinocap-4, dinocap-6, meptyldinocap, dinocton, dinopenton, dinosulfon, dinoterbon, and DNOC; a dithiocarbamate fungicide, such as azithiram, carbamorph, cufraneb, cuprobam, disulfuram, ferbam, metam, nabam, tecoram, thiram, and ziram; a cyclic dithiocarbamate fungicide, such as dazomet, etem, and milneb; a polymeric dithiocarbamate fungicide, such as mancopper, mancozeb, maneb, metiram, polycarbamate, propineb, and zineb; an imidazole fungicide, such as cyazofamid, fenamidone, fenapanil, glyodin, iprodione, isovaledione, pefurazoate, triazoxide; an inorganic fungicide, such as potassium azide, potassium thiocyanate, sodium azide, sulfur; a mercury fungicide; an inorganic mercury fungicide, such as mercuric chloride, mercuric oxide, and mercurous chloride; an organomercury fungicide, such as (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, and tolylmercury acetate; a morpholine fungicide, such as aldimorph, benzamorf, carbamorph, dimethomorph, dodemorph, fenpropimorph, flumorph, and tridemorph; an organophosphorus fungicide, such as ampropylfos, ditalimfos, edifenphos, fosetyl, hexylthiofos, iprobenfos, phosdiphen, pyrazophos, tolclofos-methyl, and triamiphos; an organotin fungicide, such as decafentin, fentin, and tributyltin oxide; an oxathiin fungicide, such as carboxin and oxycarboxin; an oxazole fungicide, such as chlozolinate, dichlozoline, drazoxolon, famoxadone, hymexazol, metazoxolon, myclozolin, oxadixyl, and vinclozolin; a polysulfide fungicide, such as barium polysulfide, calcium polysulfide, potassium polysulfide, and sodium polysulfide; a pyrazole fungicide, such as bixafen, furametpyr, isopyrazam, penflufen, penthiopyrad, pyraclostrobin, pyrametostrobin, pyraoxystrobin, rabenzazole, and sedaxane; a pyridine fungicide, such as boscalid, buthiobate, dipyrithione, fluazinam, fluopicolide, fluopyram, pyribencarb, pyridinitril, pyrifenox, pyroxychlor, and pyroxyfur; a pyrimidine fungicide, such as bupirimate, diflumetorim, dimethirimol, ethirimol, fenarimol, ferimzone, nuarimol, and triarimol; an anilinopyrimidine fungicide, such as cyprodinil, mepanipyrim, and pyrimethanil; a to pyrrole fungicide, such as fenpiclonil, fludioxonil, and fluoroimide; a quinoline fungicide, such as ethoxyquin, halacrinate, 8-hydroxyquinoline sulfate, quinacetol, quinoxyfen, and tebufloquin; a quinone fungicide, such as benquinox, chloranil, dichlone, and dithianon; a quinoxaline fungicide, such as chinomethionat, chlorquinox, and thioquinox; a thiazole fungicide, such as ethaboxam, etridiazole, isotianil, metsulfovax, octhilinone, thiabendazole, and thifluzamide; a thiazolidine fungicide, such as flutianil and thiadifluor; a thiocarbamate fungicide, such as methasulfocarb and prothiocarb; a thiophene fungicide, such as ethaboxam and silthiofam; a triazine fungicide, such as anilazine; a triazole fungicide, such as amisulbrom, bitertanol, fluotrimazole, triazbutil; a triazolopyrimidine fungicide, such as ametoctradin; an urea fungicide, such as bentaluron, pencycuron, and quinazamid; and an unclassified fungicide, such as acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, proquinazid, pyroquilon, sodium orthophenylphenoxide, spiroxamine, sultropen, thicyofen, tricyclazole, and zinc naphthenate. The at least one fungicide may also include succinate dehydrogenase inhibitors such as benzovindiflupyr, sedaxane, boscalid, fluxapyroxad, fluopyram, and penthiopyrad.

At least one insecticide may be chosen from an antibiotic insecticide, such as allosamidin and thuringiensin; an acrocyclic lactone insecticide; an avermectin insecticide, such as abamectin, doramectin, emamectin, eprinomectin, ivermectin, and selamectin; a milbemycin insecticide, such as lepimectin, milbemectin, milbemycin oxime, and moxidectin; a spinosyn insecticide, such as spinetoram and spinosad; an arsenical insecticide, such as calcium arsenate, copper acetoarsenite, copper arsenate, lead arsenate, potassium arsenite, and sodium arsenite; a botanical insecticide, such as anabasine, azadirachtin, d-limonene, nicotine, pyrethrins, cinerins, cinerin I, cinerin II, jasmolin I, jasmolin II, pyrethrin I, pyrethrin II, quassia, rotenone, ryania, and sabadilla; a carbamate insecticide, such as bendiocarb and carbaryl; a benzofuranyl methylcarbamate insecticide, such as benfuracarb, carbofuran, carbosulfan, decarbofuran, and furathiocarb; a dimethylcarbamate insecticide, such as dimetan, dimetilan, hyquincarb, and pirimicarb; an oxime carbamate insecticide, such as alanycarb, aldicarb, aldoxycarb, butocarboxim, butoxycarboxim, methomyl, nitrilacarb, oxamyl, tazimcarb, thiocarboxime, thiodicarb, and thiofanox; a phenyl methylcarbamate insecticide, such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC, and xylylcarb; a desiccant insecticide, such as boric acid, diatomaceous earth, and silica gel; a diamide insecticide, such as chlorantraniliprole, cyantraniliprole, and flubendiamide; a dinitrophenol insecticide, such as dinex, dinoprop, dinosam, and DNOC; a fluorine insecticide, such as barium hexafluorosilicate, cryolite, sodium fluoride, sodium hexafluorosilicate, and sulfluramid; a formamidine insecticide, such as amitraz, chlordimeform, formetanate, and formparanate; a fumigant insecticide, such as acrylonitrile, carbon disulfide, carbon tetrachloride, chloroform, chloropicrin, para-dichlorobenzene, 1,2-dichloropropane, ethyl formate, ethylene dibromide, ethylene dichloride, ethylene oxide, hydrogen cyanide, iodomethane, methyl bromide, methylchloroform, methylene chloride, naphthalene, phosphine, sulfuryl fluoride, and tetrachloroethane; an inorganic insecticide, such as borax, boric acid, calcium polysulfide, copper oleate, diatomaceous earth, mercurous chloride, potassium thiocyanate, silica gel, sodium thiocyanate; an insect growth regulator; a chitin synthesis inhibitor, such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron, and triflumuron; a juvenile hormone mimic, such as epofenonane, fenoxycarb, hydroprene, kinoprene, methoprene, pyriproxyfen, and triprene; a juvenile hormone, such as juvenile hormone I, juvenile hormone II, and juvenile hormone III; a moulting hormone agonist, such as chromafenozide, halofenozide, methoxyfenozide, and tebufenozide; a moulting hormone, such as a-ecdysone and ecdysterone; a moulting inhibitor, such as diofenolan; a precocene, such as precocene I, precocene II, and precocene III; an unclassified insect growth regulator, such as dicyclanil; a nereistoxin analogue insecticide, such as bensultap, cartap, thiocyclam, and thiosultap; a nicotinoid insecticide, such as flonicamid; a nitroguanidine insecticide, such as clothianidin, dinotefuran, imidacloprid, and thiamethoxam; a nitromethylene insecticide, such as nitenpyram and nithiazine; a pyridylmethylamine insecticide, such as acetamiprid, imidacloprid, nitenpyram, and thiacloprid; an organochlorine insecticide, such as bromo-DDT, camphechlor, DDT, pp′-DDT, ethyl-DDD, HCH, gamma-HCH, lindane, methoxychlor, pentachlorophenol, and TDE; a cyclodiene insecticide, such as aldrin, bromocyclen, chlorbicyclen, chlordane, chlordecone, dieldrin, dilor, endosulfan, alpha-endosulfan, endrin, HEOD, heptachlor, HHDN, isobenzan, isodrin, kelevan, and mirex; an organophosphorus insecticide; an organophosphate insecticide, such as bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP, and tetrachlorvinphos; an organothiophosphate insecticide, such as dioxabenzofos, fosmethilan, and phenthoate; an aliphatic organothiophosphate insecticide, such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, IPSP, isothioate, malathion, methacrifos, oxydemeton-methyl, oxydeprofos, oxydisulfoton, phorate, sulfotep, terbufos, and thiometon; an aliphatic amide organothiophosphate insecticide, such as amidithion, cyanthoate, dimethoate, ethoate-methyl, formothion, mecarbam, omethoate, prothoate, sophamide, and vamidothion; an oxime organothiophosphate insecticide, such as chlorphoxim, phoxim, and phoxim-methyl; a heterocyclic organothiophosphate insecticide, such as azamethiphos, coumaphos, coumithoate, dioxathion, endothion, menazon, morphothion, phosalone, pyraclofos, pyridaphenthion, and quinothion; a benzothiopyran organothiophosphate insecticide, such as dithicrofos and thicrofos; a benzotriazine organothiophosphate insecticide, such as azinphos-ethyl and azinphos-methyl; an isoindole organothiophosphate insecticide, such as dialifos and phosmet; an isoxazole organothiophosphate insecticide, such as isoxathion and zolaprofos; a pyrazolopyrimidine organothiophosphate insecticide, such as chlorprazophos and pyrazophos; a pyridine organothiophosphate insecticide, such as chlorpyrifos and chlorpyrifos-methyl; a pyrimidine organothiophosphate insecticide, such as butathiofos, diazinon, etrimfos, lirimfos, pirimiphos-ethyl, pirimiphos-methyl, primidophos, pyrimitate, and tebupirimfos; a quinoxaline organothiophosphate insecticide, such as quinalphos and quinalphos-methyl; a thiadiazole organothiophosphate insecticide, such as athidathion, lythidathion, methidathion, and prothidathion; a triazole organothiophosphate insecticide, such as isazofos and triazophos; a phenyl organothiophosphate insecticide, such as azothoate, bromophos, bromophos-ethyl, carbophenothion, chlorthiophos, cyanophos, cythioate, dicapthon, dichlofenthion, etaphos, famphur, fenchlorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3, and trifenofos; a phosphonate insecticide, such as butonate and trichlorfon; a phosphonothioate insecticide, such as mecarphon; a phenyl ethylphosphonothioate insecticide, such as fonofos and trichloronat; a phenyl phenylphosphonothioate insecticide, such as cyanofenphos, EPN, and leptophos; a phosphoramidate insecticide, such as crufomate, fenamiphos, fosthietan, mephosfolan, phosfolan, and pirimetaphos; a phosphoramidothioate insecticide, such as acephate, isocarbophos, isofenphos, isofenphos-methyl, methamidophos, and propetamphos; a phosphorodiamide insecticide, such as dimefox, mazidox, mipafox, and schradan; an oxadiazine insecticide, such as indoxacarb; an oxadiazolone insecticide, such as metoxadiazone; a phthalimide insecticide, such as dialifos, phosmet, and tetramethrin; a pyrazole insecticide, such as chlorantraniliprole, cyantraniliprole, dimetilan, tebufenpyrad, and tolfenpyrad; a penylpyrazole insecticide, such as acetoprole, ethiprole, fipronil, pyraclofos, pyrafluprole, pyriprole, and vaniliprole; a pyrethroid insecticide; a pyrethroid ester insecticide, such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, and transfluthrin; a pyrethroid ether insecticide, such as etofenprox, flufenprox, halfenprox, protrifenbute, and silafluofen; a pyrimidinamine insecticide, such as flufenerim and pyrimidifen; a pyrrole insecticide, such as chlorfenapyr; a tetramic acid insecticide, such as spirotetramat; a tetronic acid insecticide, such as spiromesifen; a thiazole insecticide, such as clothianidin and thiamethoxam; a thiazolidine insecticide, such as tazimcarb and thiacloprid; a thiourea insecticide, such as diafenthiuron; an urea insecticide, such as flucofuron, sulcofuron, and chitin synthesis inhibitors; and an unclassified insecticide, such as closantel, copper naphthenate, crotamiton, EXD, fenazaflor, fenoxacrim, hydramethylnon, isoprothiolane, malonoben, metaflumizone, nifluridide, plifenate, pyridaben, pyridalyl, pyrifluquinazon, rafoxanide, sulfoxaflor, triarathene, and triazamate.

At least one molluscicide may be chosen from a bromoacetamide, calcium arsenate, cloethocarb, copper acetoarsenite, copper sulfate, fentin, metaldehyde, methiocarb, niclosamide, pentachlorophenol, sodium pentachlorophenoxide, tazimcarb, thiacloprid, thiodicarb, tralopyril, tributyltin oxide, trifenmorph, and trimethacarb.

At least one nematicide may be chosen from an antibiotic nematicide, such as abamectin; a carbamate nematicide, such as benomyl, carbofuran, carbosulfan, and cloethocarb; an oxime carbamate nematicide, such as alanycarb, aldicarb, aldoxycarb, and oxamyl; an organophosphorus nematicide; an organophosphate nematicide, such as diamidafos, fenamiphos, fosthietan, and phosphamidon; an organothiophosphate nematicide, such as cadusafos, chlorpyrifos, dichlofenthion, dimethoate, ethoprophos, fensulfothion, fosthiazate, heterophos, isamidofos, isazofos, phorate, phosphocarb, terbufos, thionazin, and triazophos; a phosphonothioate nematicide, such as imicyafos and mecarphon; and an unclassified nematicide, such as acetoprole, benclothiaz, chloropicrin, dazomet, DBCP, DCIP, 1,2-dichloropropane, 1,3-dichloropropene, furfural, iodomethane, metam, methyl bromide, methyl isothiocyanate, and xylenols.

At least one rodenticide may be chosen from a botanical rodenticide, such as scilliroside and strychnine; a coumarin rodenticide, such as brodifacoum, bromadiolone, coumachlor, coumafuryl, coumatetralyl, difenacoum, difethialone, flocoumafen, and warfarin; an indandione rodenticide, such as chlorophacinone, diphacinone, and pindone; an inorganic rodenticide, such as arsenous oxide, phosphorus, potassium arsenite, sodium arsenite, thallium sulfate, and zinc phosphide; an organochlorine rodenticide, such as gamma-HCH, HCH, and lindane; an organophosphorus rodenticide, such as phosacetim; a pyrimidinamine rodenticide, such as crimidine; a thiourea rodenticide, such as antu; a urea rodenticide, such as pyrinuron; and an unclassified rodenticide, such as bromethalin, chloralose, a-chlorohydrin, ergocalciferol, fluoroacetamide, flupropadine, hydrogen cyanide, norbormide, and sodium fluoroacetate. At least one virucide may include ribavirin. This list is exemplary of course.

In exemplary agricultural embodiments, the AI may include at least one of azoxystrobin, abamectin, ametryn, acetochlor, atrazine, benoxacor, chlorothalonil, emamectin, fludioxonil, chlorothalonil, metalaxyl, pinoxaden, thiamethoxam, and abamectin.

### EXAMPLES

In order that those skilled in the art will be better able to practice embodiments of the invention, the following examples are given by way of illustration and not by way of limitation. In the following examples, as well as elsewhere in the specification and claims, temperatures are in degrees Celsius, and the pressure is atmospheric unless indicated otherwise.

### Example 1

### Generation of Phage Particles Having Binding Domains Selected to Bind to AI Solid Particles

### 1.1 Materials.

A commercial phage library (Ph.D.-7, New England Biolabs, Ipswich, Mass.) was used. Other reagents, including polyethylene glycol (Mw=8,000 g/mol), Tris-HCl, glycine-HCl (Trizma® hydrochloride, T6666), NaCl (BioReagent 99.5%, S5886), sodium iodide (ACS reagent 99.5%, 383112), tween-20 (P9416), and ethanol (ACS reagent ≧99.5%, 459844) were obtained from Sigma-Aldrich and used as received (St. Louis, Mo.). 5-bromo-4-chloro-3indolyl-β-D-galactopyranoside (X-gal, AC327241000) was purchased from Acros Organics (Morris Plains, N.J.). Isopropyl-β-D-thiogalactopyranoside (IPTG, BP1755) and bovine serum albumin (BSA, BP1600) were purchased from Fisher Scientific (Pittsburgh, Pa.). Thiamethoxam (TMX) was provided by Syngenta Crop Protection (Greensboro, N.C.).

### 1.2 Phage Display Screening for Selection of TMX Binding Peptide Motifs.

Biopanning using the phage library was performed against thiamethoxam. The initial phage library solution was diluted by adding 10 μl of library (˜1×10¹³ plaque forming units/ml or pfu/ml) to 1 ml of 0.1% TBST buffer (50 mM Tris-HCl pH 7.5, 150 mM NaCl, 0.1% v/v tween-20). TMX was treated with blocking solution (0.1 M NaHCO₃ pH 8.6, 5 mg/ml BSA) and washed several times with 0.1% TBST and dispersed into 200 μl TBS to a final concentration of 10 mg/ml. 100 μl of the diluted phage library was then introduced into the TMX solution and incubated for 1 h with gentle rocking. The sample was then washed several times with TMX saturated 0.1% TBST buffer and transferred into the new centrifuge tubes. This step removes nonspecific binding phages or any phages with a strong affinity to the polypropylene centrifuge tubes. Next, to measure the number of phage adsorbed to the TMX, phage were eluted from the TMX surface by incubating with 100 μl of 0.2 M glycine-HCl (pH 2.2), 1 M BSA solution, which was then neutralized with 15 μl of 1 M Tris base (pH 9.0). The eluted phage were amplified with 20 ml of 1:100 diluted overnight culture of E. coli (ER 2738) that was grown in LB media at 37° C. for 4.5 h and purified through polyethylene glycol precipitation. These panning procedures were repeated three times, using increasing tween-20 concentration in each round (0.3, 0.5% v/v) to increase the stringency of binding to the TMX target.

The final eluted phage solutions were serially diluted and quantified by plating on agar containing 5-bromo-4-chloro-3indolyl-β-D-galactopyranoside (X-Gal) and isopropyl-β-D-thiogalactopyranoside (IPTG) (LB/IPTG/X-Gal plates), which show blue plaques after incubating at 37° C. overnight. Individual blue plaques were selected and separately amplified in 1 ml of 1:100 diluted overnight culture of E. coli in LB media at 37° C. for 4.5 h. From these amplified phage stock, phage were separately precipitated.

### 1.3 Characterization of TMX-Phage Complex.

Atomic force microscopy (AFM) images of M13 bacteriophage on a mica substrate were obtained using a multimode AFM in tapping mode under ambient conditions (Nanoscope III, Bruker Inc., Santa Barbara, Calif.). First, M13 bacteriophage solution was placed onto a cleaved mica substrate. After incubating for 30 min, the mica surface was washed with distilled water, air dried and then analyzed by AFM. Height, amplitude and phase signals were simultaneously recorded. The morphology of M13 bacteriophage on the mica substrate was imaged using tapping mode at 1.5 Hz scan rate. AFM microscopy (Bruker Inc., Bioscope II mounted with Zeiss Axiovert 200 inverted light microscope) was also performed to measure the unbinding forces between M13 bacteriophage and TMX. To attach M13 bacteriophage to the silicon nitride AFM tips (DNP-10, Bruker Inc., Santa Barbara, Calif.), were first cleaned in a piranha solution (70% H₂SO₄/30% H₂O₂ v/v). Following washing, a silanizing agent, 4% 3-amino-propyltriethoxysilane (APTES, 440140, Sigma Aldrich) in ethanol was used to covalently attach amines on the tip incubated for 1 hr at room temperature. Silanized tips were subsequently treated with 2.5% glutaraldehyde (GA, Ted Pella) for 10 min to facilitate phage immobilization. The surface was then rinsed against pure deionized water and then incubated with M13 bacteriophage for 1 h. The TMX sample was affixed to a glass substrate using epoxy resin. The prepared samples were mounted into the AFM liquid flow cell. Experiments were performed in TBS buffer at room temperature. A Bruker tip with 0.12 N/m spring constant was used for measuring the interaction between M13 bacteriophage and TMX. The measurements were performed at a 2.06 μm/s retraction rate. Force-distance curves were collected at three randomly chosen points on the TMX surface. At least 1,000 force measurements were made at each location.

TMX-M13 bacteriophage complex were characterized with electron microscopy. To image the bound phage, samples dissolved in TBS solution were applied to a poly-L-lysine coated glass coverslip and then completely air dried in a fume hood for approximately 2-3 h. To prevent electron charging, Au was sputtered on the sample. Field-emission scanning electron microscopy (FE-SEM) images were taken using a scanning microscope (Hitachi 4700 FE-SEM) at a 5 kV accelerating voltage. For transmission electron microscopy (TEM), a drop of TMX-M13 complex solution was added onto poly-L-lysine coated 200 mesh formvar-carbon coated copper grids. The grids were rinsed three times with water and then negative stained with 0.5% aqueous uranyl acetate. TEM images of TMX-phage complex were obtained with a transmission electron microscope (Zeiss LIBRA 120) at a 120 kV accelerating voltage.

The TMX-M13 complex was characterized with confocal laser scanning microscopy (5 live duo, Zeiss, Address) using the 488 nm vacuum wavelength emission line of an argon ion laser. To confirm the TMX surfaces were bound with M13 bacteriophage, TMX-M13 complexes were incubated for 1 h with AIexa Fluor® 488 tagged-anti M13 monoclonal antibody and centrifuged for 30 min to remove free dye molecules. The fluorescence image was taken using emission between 505 nm and 550 nm.

The surface charge of thiamethoxam (TMX) was measured by characterizing the zeta potential (ZetaPALS, Brookehaven Instruments). For each sample measured, 10 mg of TMX was suspended in 1 mL of Tris-buffered saline (TBS) solution at pH 7.5.

### Example 3

### Selection of Other Binding Domains

The identification and characterization of binding domains capable of binding to solid particles of an active ingredient may also be achieved using phage display of alternative polypeptide structures to that described in Example 1, for example, using protein structural domains that are capable of forming contacts with target surfaces to achieve affinity-interactions. Such protein structural domains may include FAb; Fv; scFv; stAb; dAb; V_{HH}; IgNAR; CDRs; DARPin ankyrin-repeat proteins; anti-calins; antibody-mimics, or fragments thereof. In addition, phage-display libraries may be created from naive or immune binding domain molecular repertoires. Naive repertoires may be generated from e.g. un-immunized animal B-lymphocyte mRNA and/or diversity-expanded DNA libraries through the use of PCR and degenerate oligonucleotides. Immune repertoires may be generated by first immunizing an animal with an appropriate formulation of crystalline particles, monitoring for an immune response and, if a response is evident, preparing B-lymphocyte mRNA from which PCR can be used to amplify the desired molecular repertoire for cloning into a bacteriophage-display library. The phage-display library may be incubated in solution with the target surface for a time period and target-specific bacteriophage particles may be selected by removing unbound bacteriophage (by solution exchange for example), and replicating those bacteriophage that have remained bound to the target. Target-specific bacteriophage can be DNA-sequenced to determine the exact nucleic acid code for the binding-domain, allowing further options for engineering/improvement of the binding-domain, or use independently of the bacteriophage itself. Alternative binding-domain display technologies, e.g. bacterial, yeast, ribosomal, may be employed in the selection of desired binding-domains.

### Example 4

### Influence on Crystal Shape

An aqueous solution of TMX at a concentration of 11 mg/ml was prepared. The respective portion of TMX and water was added to an appropriately sized vessel and heated to 60° C. to dissolve the TMX. Once dissolved, phage particles having binding domains selected to bind to TMX were added to the achieve target loading (phage particles having binding domains concentration C=37 mg/ml). Phage loading was a C, C/2, C/10, C/25, and C/100. The solution was allowed to cool to room temperature and aged until crystallization was observed. Crystalline products were separated from the liquor by filtration and characterized by microscopy.

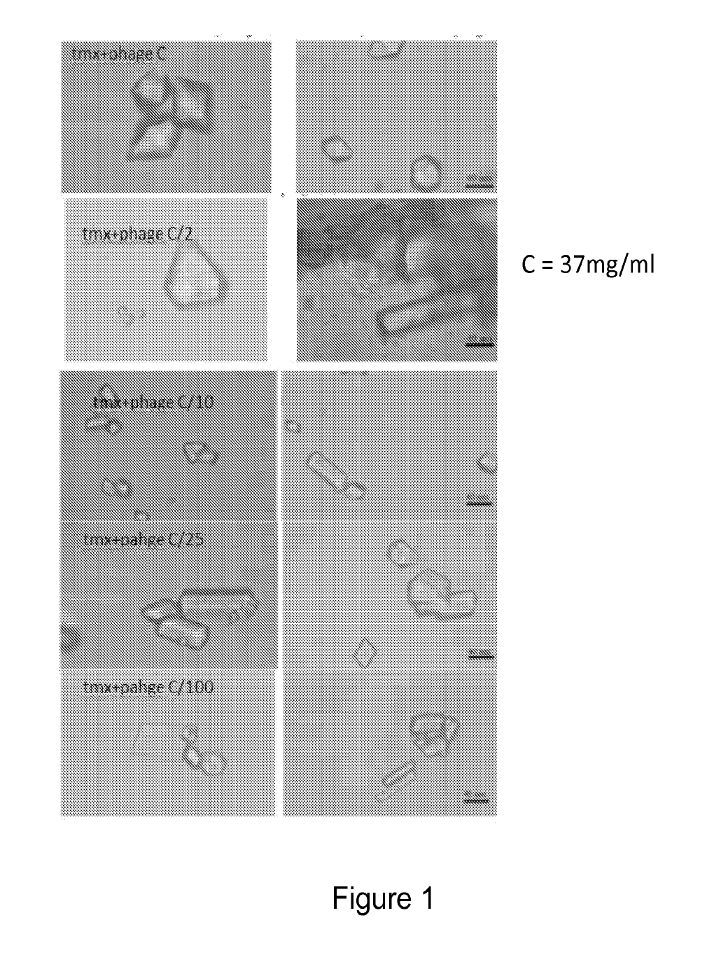

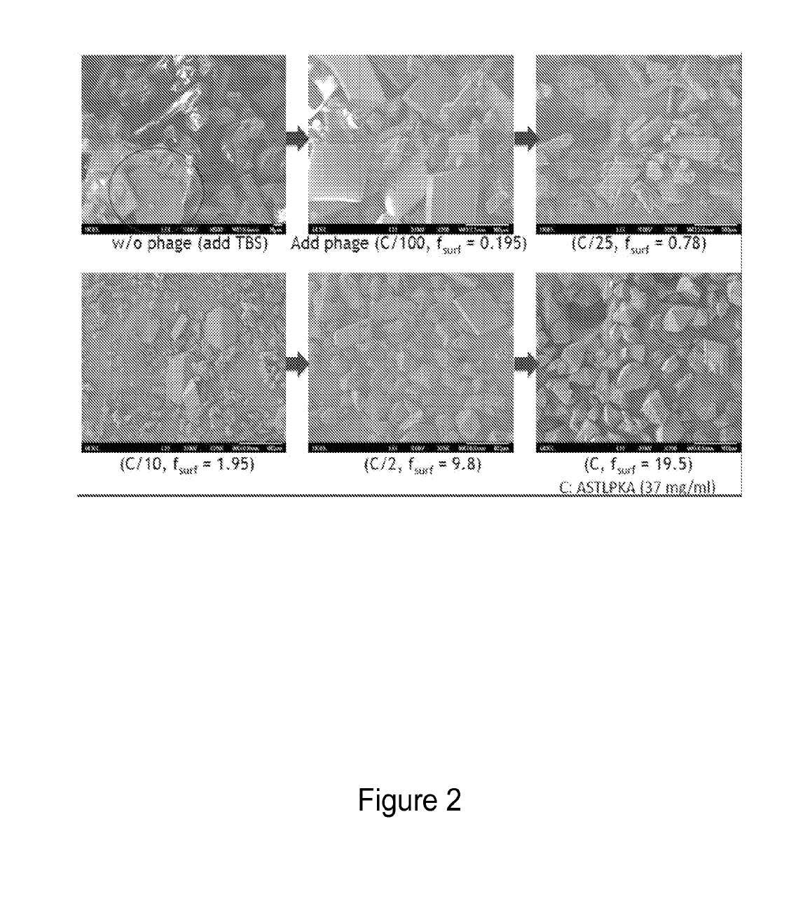

FIG. 1 shows optical microscopy images of TMX crystallized in the presence of different concentrations of phage particles having binding domains selected to bind to TMX. FIG. 2 shows electron microscopy images of TMX crystallized in the presence of different concentrations of phage particles having binding domains selected to bind to TMX. As seen in both the optical and electron microscopy images, at increasing concentrations, e.g. C/2 to C, phage particles having biding domains selected to bind to TMX alter the shape of the TMX crystals to form a more bi-pyramidal shape.

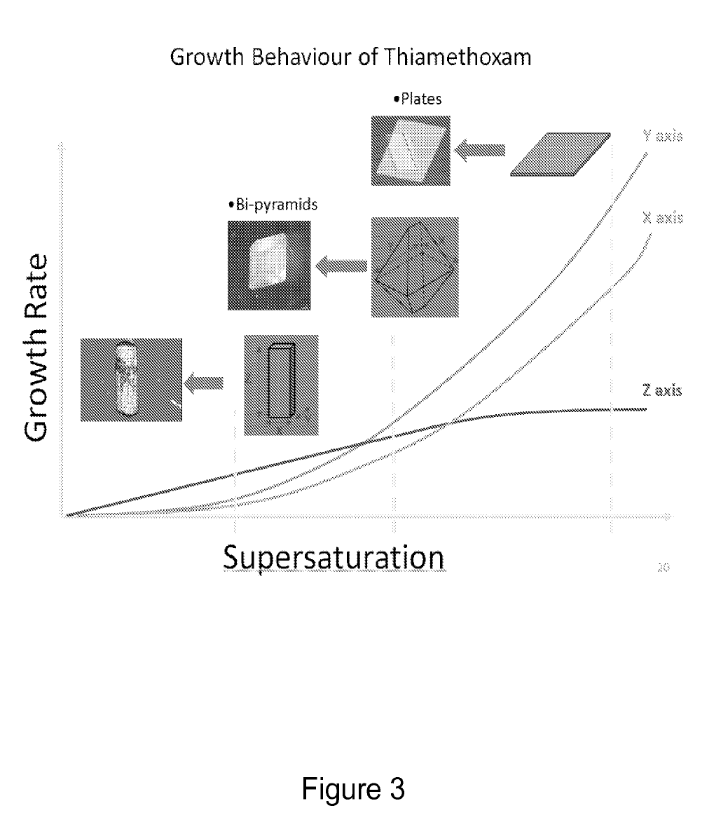

FIG. 3 illustrates different facets of a TMX crystal and exemplifies shape as a function of relative facet length.

### Example 5

### Crystal Size

An aqueous solution of TMX at a concentration of 11 mg/ml was prepared. The respective portion of TMX and water was added to an appropriately sized vessel and heated to 60° C. to dissolve the TMX. Once dissolved, phage particles having binding domains selected to bind to TMX were added to the achieve target loading set forth in the Table 1 below.

TABLE 1

| | | | | | phage |
|---|---|---|---|---|---|
| | Volume | | | | con |
| | of TMX | Volume | Concen- | Phage | com- |
| | solution | of Phage | tration | Concen- | pared |
| | (ml) | (ml) | of TMX | tration | with C |
| Sample 1 | 5 | 0 | 11 mg/ml | 0 | 0 |
| (TMX | | | | | |
| control) | | | | | |
| Sample 2 | 3.34 | 1.66 ml | 11 mg/ml | 1.48 mg/ml | C/25 |
| (TMX + | | | | (7.4 mg) | |
| Phage) | | | | | |
| Sample 3 | 4.585 | 0.415 ml | 11 mg/ml | 0.37 mg/ml | C/100 |
| (TMX + | | | | (1.85 mg) | |
| Phage) | | | | | |

The solution was allowed to cool to room temperature and aged until crystallization was observed. Crystalline products were separated from the liquor by filtration and particle size distribution was measured using a Malvern Mastersizer.

The results of the product size distribution are contained in Table 2 below.

TABLE 2

| | Size (μm) | | | | | | | |
|---|---|---|---|---|---|---|---|---|
| Sample | Dv (0.5) | | | average | Dv (0.95) | | | average |
| TMX | 23.378 | 22.048 | 23.891 | 23.1057 | 52.18 | 54.16 | 55.74 | 54.11 |
| control | | | | | | | | |
| C/25 | 14.160 | 15.677 | 16.634 | 15.502 | 35.97 | 38.06 | 37.69 | 36.94 |
| C/100 | 13.542 | 12.746 | 13.267 | 13.149 | 33.8 | 29.73 | 28.72 | 30.75 |

As seen in Table 2, the particle size data in the table above show a marked reduction in the particle size of TMX crystallized in the presence of phage particles having binding domains selected to bind to TMX.

### Example 6

### Improved Desupersaturation

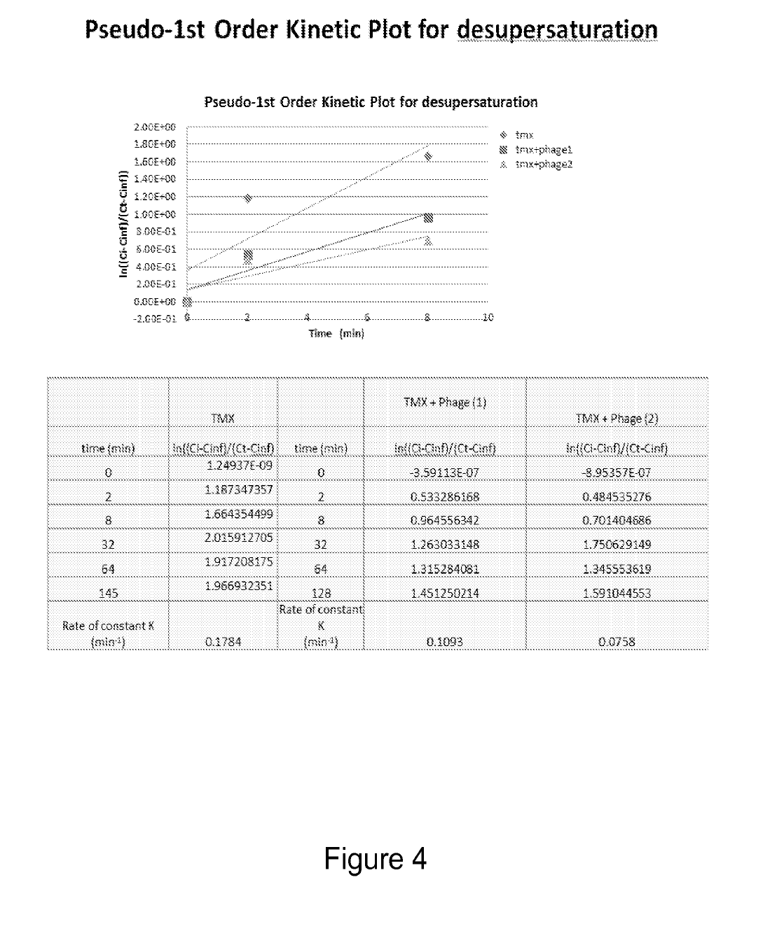

Crystallization kinetics experiments were conducted in a 50-mL round bottom flask. 0.44 g TMX was dissolved in 40 ml water (1.1% TMX) at 50° C. prior to cooling to the desired temperature (37° C.) to obtain a supersaturated solution. The mixture was seeded with 50% TMX technical. Periodic slurry samples were taken and immediately filtered using preheated filtration apparatus and diluted with water for analysis by HPLC. The effect of phage particles having binding domains selected to bind to TMX was measured by mixing phage particles having binding domains selected to bind to TMX (concentration approximately 10% with respect to the total amount of TMX) at elevated temperature and following the same procedure. Results are illustrated in FIG. 4.

Numerous characteristics and advantages have been set forth in the foregoing description, together with details of structure and function. The disclosure, however, is illustrative only, and changes may be made in detail, especially in matters of performance of the method steps within the principle of the disclosure. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein, and every number between the end points. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10, as well as all ranges beginning and ending within the end points, e.g. 2 to 9, 3 to 8, 3 to 9, 4 to 7, and finally to each number 1, 2, 3, 4, 5, 6, 7, 8, 9 and 10 contained within the range.

1. A method of producing active ingredient (AI) solid particles, the method comprising:
creating a mother liquor comprising an active ingredient (AI) and a solvent by dissolving the AI in the solvent;
adding to the mother liquor a concentration of phage particles having binding domains, or binding portions thereof, selected to bind to the AI solid particles;
precipitating the AI solid particles from the mother liquor; and
separating the precipitated AI solid particles from the mother liquor.

2. The method of claim 1, comprising at least one of
heating the AI and the solvent when creating the mother liquor;
reacting at least two precursors to synthesize the AI in the solvent;
adding an antisolvent to the mother liquor to facilitate precipitation of the AI solid particles,
evaporating the solvent from the mother liquor to facilitate precipitation of the AI solid particles; and
cooling the mother liquor to facilitate precipitation of the AI solid particles.

3. The method of claim 1, wherein precipitating the AI solid particles from the mother liquor further comprises
seeding the mother liquor with a plurality of seed AI particles.

4. The method of claim 3, wherein the plurality of seed AI particles are added at at least one of
a concentration in the range of 0.02% to 10%; and
a temperature in the range of 20° C. to 60° C.

5. The method of claim 1, wherein creating the mother liquor includes creating an AI concentration is in the range of 5 wt % to 70 wt %.

6. The method of claim 5, wherein the concentration of phage particles having binding domains, or binding portions thereof, is in the range of 0.01% to 75% of the AI concentration.

7. The method of claim 6, wherein the concentration of phage particles having binding domains, or binding portions thereof, is in the range of 10% to 30% of the AI concentration.

8. The method of claim 1, wherein the dissolved AI includes a pesticidal compound chosen from at least one of an acaricide, an algicide, an avicide, a bactericide, a fungicide, a herbicide, an insecticide, a molluscicide, a nematicide, a rodenticide, and a virucide.

9. The method of claim 1, wherein the dissolved AI includes at least one compound chosen from an amide herbicide, a chloroacetanilide herbicide, a cyclopropylisoxazole herbicide, a nitrophenyl ether herbicide, an imidazolinone herbicide, an organophosphorus herbicide, a phenylpyrazole herbicide, an amide fungicide, an anilide fungicide, a strobilurin fungicide, a carbanilate fungicide, a triazole fungicide, a copper fungicide, a pyrazole fungicide, a pyridine fungicide, an anilinopyrimidine fungicide, a pyrrole fungicide, a thiazole fungicide, an avermectin insecticide, a diamide insecticide, a nicotinoid insecticide, a nitroguanidine insecticide, an organochlorine insecticide, a pyrazole insecticide, a pyrethroid insecticide, and a thiazole insecticide.

10. The method of claim 1, wherein the dissolved AI includes at least one of azoxystrobin, abamectin, ametryn, acetochlor, atrazine, benoxacor, chlorothalonil, emamectin, fludioxonil, chlorothalonil, metalaxyl, pinoxaden, thiamethoxam, and abamectin.

11. The method of claim 1, wherein the precipitated AI solid particles have a mean diameter in the range of about 10⁻⁸ to about 10⁻⁴ m.

12. The method of claim 1,
wherein the precipitated AI solid particles include a first crystalline growth site; and
wherein adding includes adding phage particles having binding domains, or binding portions thereof, selected to bind to the first crystalline growth site, whereby during precipitation, the phage particles having binding domains, or binding domains thereof, inhibit crystal growth at the first crystalline growth site, thereby creating a second shape (S2) that differs from a first shape (S1), wherein S1 is the shape created by a process as defined in claim 1, without the addition of phage particles having binding domains, or binding portions thereof, to the mother liquor.

13. The method of claim 12, wherein S1 defines a geometric shape including at least one facet (F1) and wherein the S2 defines a geometric shape including at least one different facet (F2).

14. The method of claim 1, further comprising
combining the precipitated and separated AI solid particles with at least one non-active ingredient chosen from a surfactant, a diluent, a wetting agent, an adhesive, a binding agent, a safener, an antifoaming agent, and an emulsifier.

15. The method of claim 1, wherein the precipitated and separated AI solid particles are combined with the at least one non-active ingredient, without additional milling of the precipitated and separated AI solid particles prior to combining.

16. The method of claim 12, wherein F2 is greater than F1 by a factor in the range of 1.5× to 5×, further including shearing the S2 and combining the sheared S2 with at least one non-active ingredient.

17. (canceled)

18. The method of claim 1, with the proviso that the mother liquor does not include at least one of, at least two of, at least three of, or at least four of the following non-active ingredients: a surfactant, a diluent, a wetting agent, an adhesive, a binding agent, a safener, an antifoaming agent, and an emulsifier.

19. A mother liquor for producing active ingredient (AI) solid particles, the mother liquor comprising:
a solvent;
an AI concentration is in the range of 5 wt % to 70 wt %; and
a concentration of phage particles having binding domains, or binding portions thereof, selected to bind to the AI, wherein the concentration of phage particles having binding domains, or binding portions thereof, is in the range of 10% to 30% of the AI concentration.

20. (canceled)

21. The mother liquor of claim 19, with the proviso that the mother liquor does not include at least one of, at least two of, at least three of, or at least four of the following non-active ingredients: a surfactant, a diluent, a wetting agent, an adhesive, a binding agent, a safener, an antifoaming agent, and an emulsifier.

22. Solid AI particles for using to make a formulated product, wherein the solid AI particles are formed by the method of claim 1, wherein the solid AI particles include a plurality of phage particles having binding domains, or binding portions thereof, selected to bind to the AI.
